(19) United States
(12) Patent Application Publication
(10) Pub. No.: US 2020/0214876 A1
(43) Pub. Date: Jul. 9, 2020

(54) Menstrual cup

(71) Applicant: Pei-Ying Tsai, New Taipei City (TW)

(72) Inventor: Pei-Ying Tsai, New Taipei City (TW)

(21) Appl. No.: 16/824,721

(22) Filed: Mar. 20, 2020

(51) Int. Cl. A61F 5/455; A61F 5/44

(52) U.S. Cl. CPC A61F 5/4553; A61F 5/4404

(57) ABSTRACT

A flexible menstrual cup includes a cup including an internal space having an opening, an outer side wall, and an inner side wall; and a stem. The opening is provided with an overflow stopping convex ring. A top surface of the overflow stopping convex ring is a bevel face. An inner side wall is provided with a plurality of vertical grooves.

### BACKGROUND OF THE INVENTION

### 1. Field of the Invention

The invention relates to personal sanitary products, and more particularly to a menstrual cup having improved characteristics and being adapted to collect menstrual fluid.

### 2. Description of Related Art

A conventional sanitary napkin structure is mostly a pad structure. The sanitary napkin is put at the top surface of the female's drawers, and the attachment wings outside the pad are reversely attached to the bottom of drawers. The pad contains a water absorbing layer made of water absorbing material, which can absorb the effluent menses in menstrual period. There are sidewalls on both sides of the pad for preventing side leakage of menses, it is the sanitary accessory used by most females in menstrual period. However, the sanitary napkin has many defects. The user will have a foreign body sensation, the user is likely to have a sense of insecurity in active state, worrying about the offset of sanitary napkin, and she is likely to have a feeling of stuffiness when it is hot. If it is not changed frequently, the user may worry about peculiar smell or infection, and she will worry about seepage when the menses volume is large, which not only fouls the clothing, but also embarrasses the user. If the user goes out without a sanitary napkin, buying a new one will increase the expense and make the user nervous. In addition, the sanitary napkin shall be changed frequently in menstrual period, a user consumes 35-40 sanitary napkins on average in a menstrual period, the quantity of refuse is increased, not friendly to the environment, and improper discarding is likely to cause contaminants, and the cost of sanitary napkin is one of the females' fixed charges. Furthermore, the users will worry about the damping of the stored sanitary napkins and the damp sanitary napkins will be unavailable.

Another conventional sanitary cotton sliver is a strip cotton. The sanitary cotton sliver is invasive, put in the vagina through two steps. The outer case is pulled out after it is put in the vagina, the female will have foreign body sensation twice. The sanitary cotton absorbs water and expands, if it absorbs menses, it expands in the vagina, and it is stuck in the vagina, so that the menses will not flow out. However, the sanitary cotton sliver has the same defects in sanitary napkin. The two-step operation is more complicated than sanitary napkin, the user will have a foreign body sensation, worries about peculiar smell or infection if it is not changed frequently, and worries about the seepage when the menses volume is large, which not only fouls the clothing, but also embarrasses the user. If the user goes out without a sanitary cotton sliver, buying a new one not only increases the expense, but also tenses the user. In addition, the sanitary cotton sliver shall be changed frequently in menstrual period, the quantity of refuse is increased, not friendly to the environment, and improper discarding is likely to cause contaminants. The users will worry about the damping of the stored sanitary cotton sliver, the damp sanitary cotton slivers will be unavailable.

Thus, the need for improvement still exists.

### SUMMARY OF THE INVENTION

It is therefore one object of the invention to provide a menstrual cup, comprising a cup including an internal space having an opening, an outer side wall, and an inner side wall; and a stem; wherein the cup is flexible; and wherein the menstrual cup is used in the menstrual period of a female, when the menstrual cup is inserted into the female's vagina, the menses can flow into the internal space through the opening, and the menstrual cup is pulled out of the vagina through the stem.

The above and other objects, features and advantages of the invention will become apparent from the following detailed description taken with the accompanying drawings.

### BRIEF DESCRIPTION OF THE DRAWINGS

FIG. 1 is a perspective view of a menstrual cup of the invention;

FIG. 2 is a longitudinal sectional view of the menstrual cup;

FIG. 3 is a side elevation of a portion of the menstrual cup;

FIG. 4 is an enlarged view of an upper portion of FIG. 2;

FIG. 5 is a fragmentary view of FIG. 2 showing the menstrual cup being flexibly compressed in response to impact; and

FIG. 6 schematically depicts the menstrual cup inserted into the vagina during menstruation for collecting menstrual fluid.

### DETAILED DESCRIPTION OF THE INVENTION

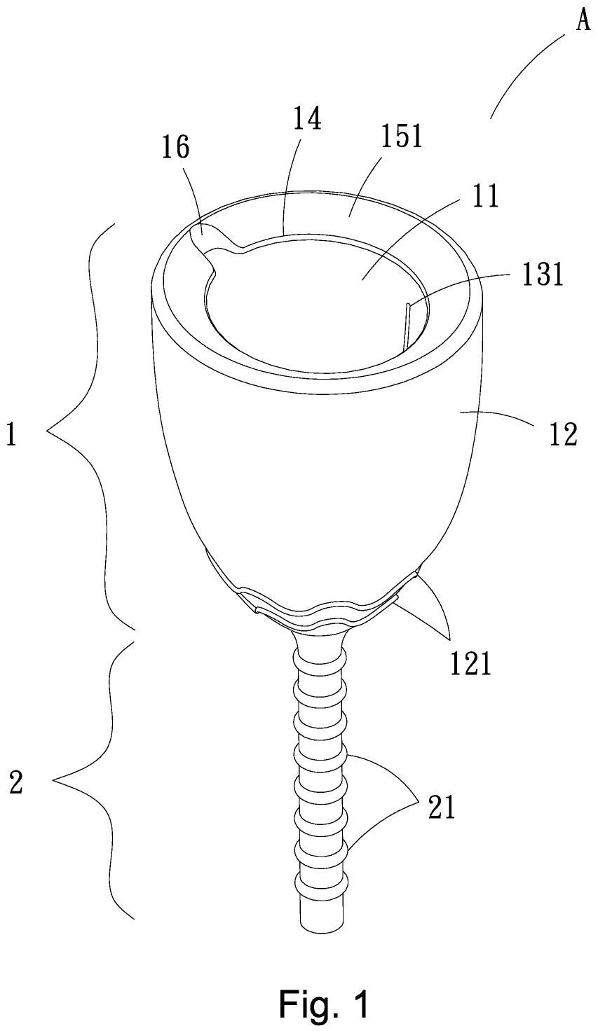

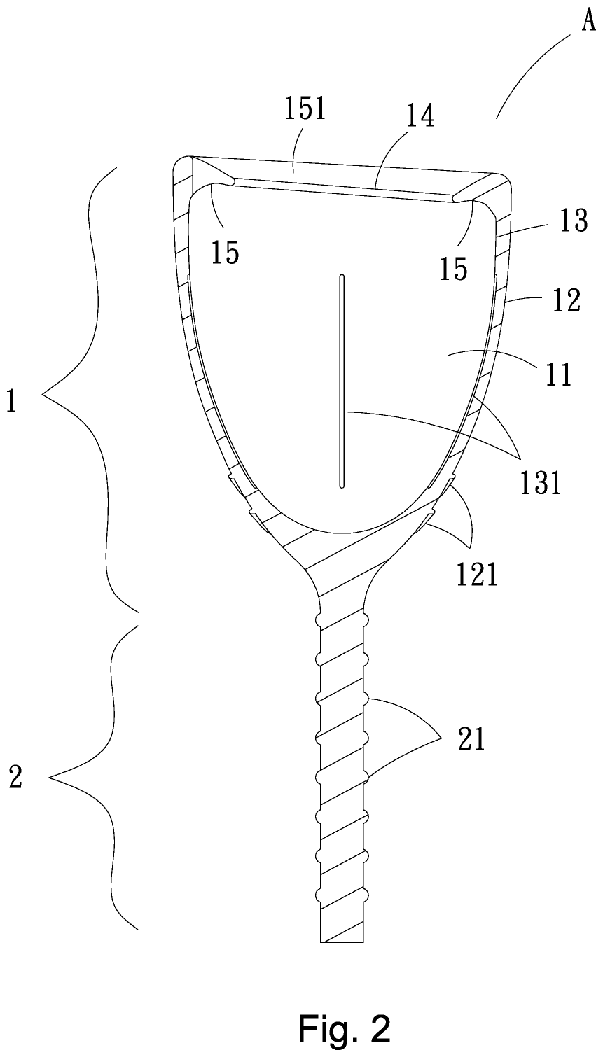

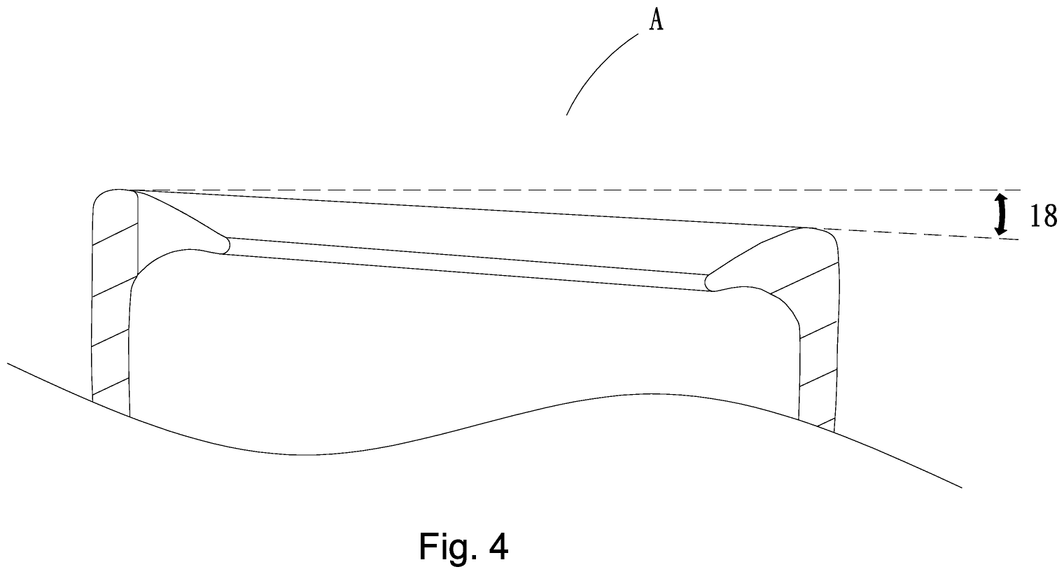

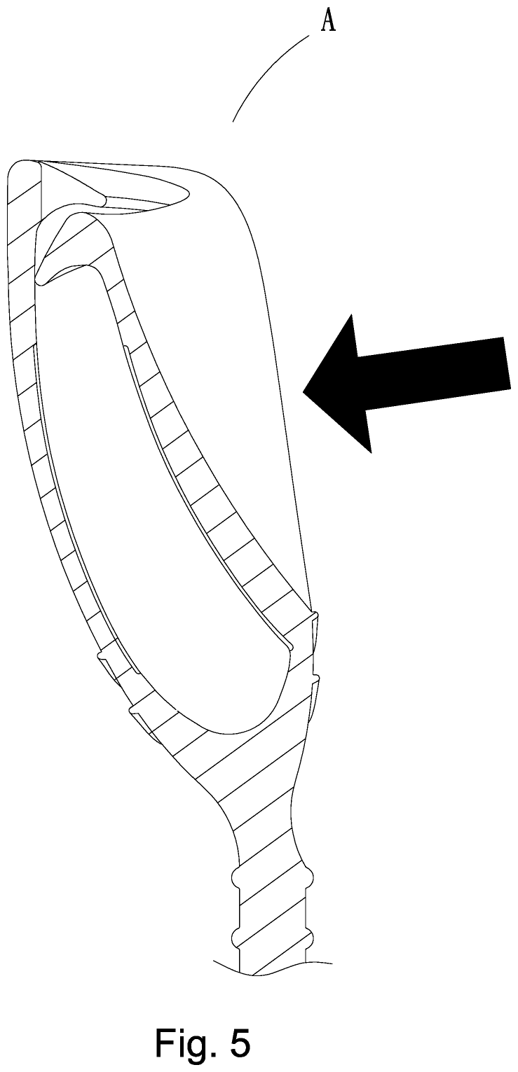

Referring to FIGS. 1 to 6, a menstrual cup A of the invention is shown. The menstrual cup A comprises a cup 1 and a stem 2. The inside of the cup 1 is an internal space 11. The cup 1 has an outer side wall 12 and an inner side wall 13. The outer side wall 12 of the cup 1 is provided with surrounding bearing ribs 121, the inner side wall 13 is provided with a plurality of grooves 131. A plurality of grooves 131 is vertical grooves. The top surface of the cup 1 is provided with an opening 14. The opening 14 is provided with an overflow stopping convex ring 15. The top surface of the overflow stopping convex ring 15 is a bevel face 151. The opening 14 is provided with a dumping port 16, the dumping port 16 is provided with a joint opening 17. The stem 2 is provided with a plurality of slip resistant convex rings 21. The stem 2 can be shortened according to user demand. The top surface of the cup 1 has a slope 18.

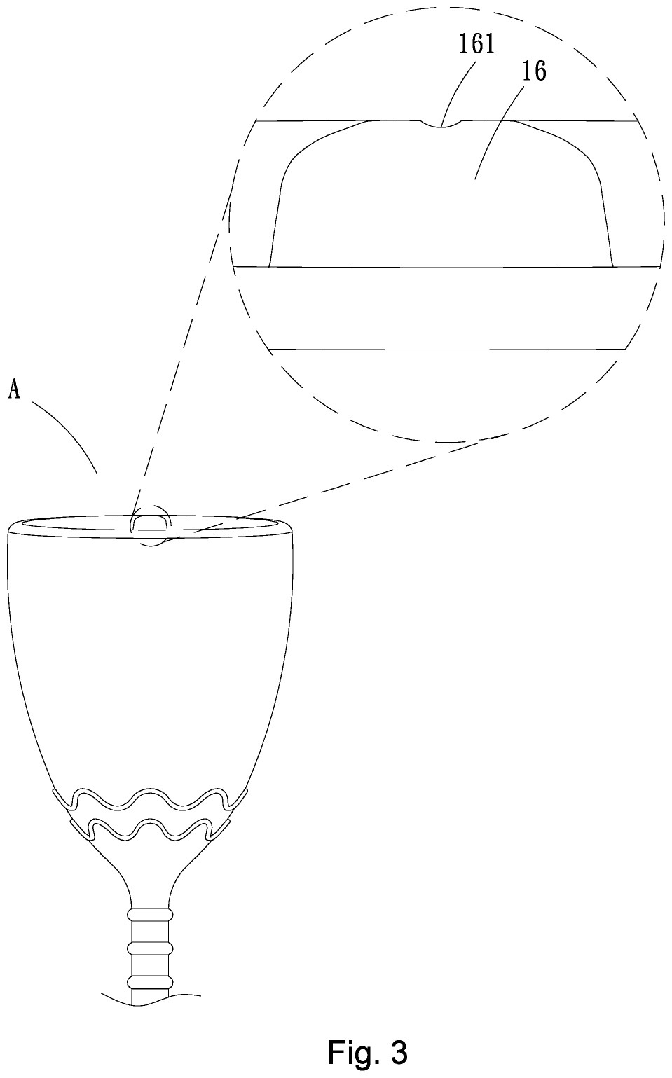

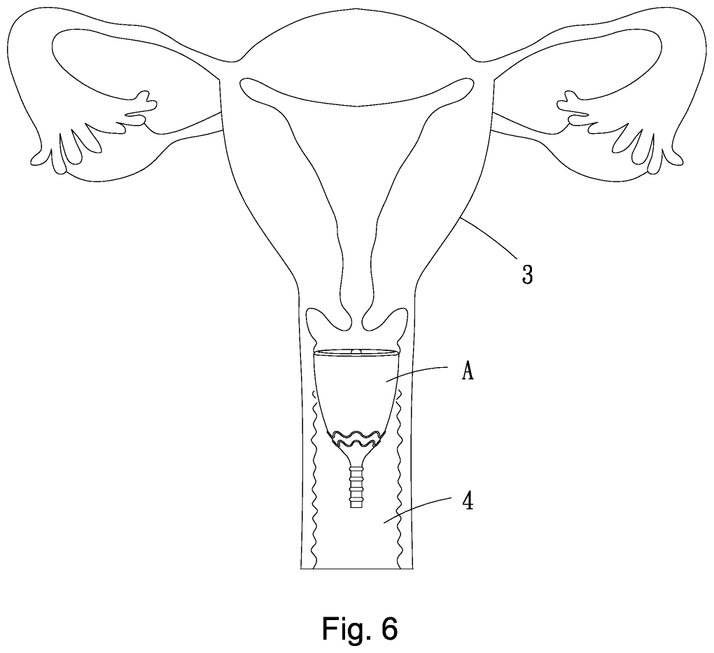

Referring to FIGS. 3 and 6 specifically, the menstrual cup A is flexible. The menstrual cup A is used in the females' menstrual period. The cup 1 can be folded by using the plurality of grooves 131 as creases. In addition, due to the slope 18 of the top surface of the cup 1, after the cup 1 is folded, the overflow stopping convex rings 15 will not contact each other, they are vertically overlapped, the volume of the cup 1 is reduced a lot, the user can grip the surrounding bearing ribs 121 to put the cup in the vagina 4. When the menstrual cup A is put in the vagina 4, the user can let go of hand, the pressed cup 1 is restored, the plurality of grooves 131 can buffer the tension when the cup 1 opens, reducing the user's discomfort. As the cup 1 is flexible, and the vagina 4 itself is elastic, the opened cup 1 will adhere to the vagina 4 wall, and the cup 1 and the vagina 4 form a vacuum state.

When the menses is discharged from the womb 3, the internal space 11 of the cup 1 can receive the menses. As the top surface of the overflow stopping convex ring 15 is the bevel face 151, which assists the menses to slide into the internal space 11 through the opening 14. When the menstrual cup A has reached the predetermined service time, the user can pull the stem 2 to tilt the cup 1 slightly, the joint opening 17 relieves vacuum state, the user supports the surrounding bearing ribs 121 with hand to take out the menstrual cup A stably. The overflow stopping convex ring 15 prevents the menses from splashing when the user takes out the menstrual cup A unsteadily. The menses can be spilled out through the dumping port 16. The menstrual cup A can be reused after it is cleaned thoroughly.

Generally speaking, the menstrual cup A provides a better option of personal sanitary product for females in menstrual period, and the invention is made of safe silica gel, which is flexible, and it can reused after it is cleaned. It meets the concept of environmental protection, reduces the quantity of refuse, and saves the cost for users. The menstrual cup A is in vacuum state when it is in operation, the menstrual cup A will not shift, and there is no sense of stuffiness. It is put in the vagina 4 in advance, the user will not forget to take it when going out. When it is put aside, as long as it is cleaned, it will not deteriorate, the defects in the known technology are remedied greatly.

While the invention has been described in terms of preferred embodiments, those skilled in the art will recognize that the invention can be practiced with modifications within the spirit and scope of the appended claims.

What is claimed is:

1. A menstrual cup, comprising:
a cup including an internal space having an opening, an outer side wall, and an inner side wall; and
a stem;
wherein the cup is flexible; and
wherein the menstrual cup is used in the menstrual period of a female, when the menstrual cup is inserted into the female's vagina, the menses can flow into the internal space through the opening, and the menstrual cup is pulled out of the vagina through the stem.

2. The menstrual cup of claim 1, wherein the opening is provided with an overflow stopping convex ring.

3. The menstrual cup of claim 2, wherein a top surface of the overflow stopping convex ring is a bevel face.

4. The menstrual cup of claim 1, wherein the opening is provided with a dumping port.

5. The menstrual cup of claim 4, wherein the dumping port is provided with a joint opening.

6. The menstrual cup of claim 5, wherein the inner side wall is provided with a plurality of grooves.

7. The menstrual cup of claim 6, wherein the plurality of grooves are vertical grooves.

8. The menstrual cup of claim 6, wherein the stem is provided with a plurality of slip resistant convex rings.

9. The menstrual cup of claim 7, wherein the outer side wall of the cup is provided with a plurality of surrounding bearing ribs.

10. The menstrual cup of claim 8, wherein a top surface of the cup has a slope.
